(19) United States
(12) Patent Application Publication
(10) Pub. No.: US 2005/0095219 A1
(43) Pub. Date: May 5, 2005

(54) Compositions for promoting vaginal cell proliferation and maturation

(76) Inventors: Shu-Ping Yang, Alpharetta, GA (US); Yanbin Huang, Roswell, GA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/696,547

(22) Filed: Oct. 29, 2003

(51) Int. Cl. A61K 31/74; A61K 31/728

(52) U.S. Cl. 424/78.26; 514/54

(57) ABSTRACT

The invention provides hyaluronic acid compounds and derivatives thereof for increasing vaginal cell growth, vaginal cell maturation and vaginal moisture, as well as compositions, articles and methods for treating and preventing vaginal conditions characterized by poor vaginal cell growth, low vaginal cell differentiation and low vaginal moisture.

### FIELD OF THE INVENTION

The invention relates to the use of compositions containing hyaluronic acid compounds to promote vaginal cell proliferation and maturation.

### BACKGROUND OF THE INVENTION

Vaginal atrophy is a common and well-recognized problem in menopausal women. It is found in up to 50% post-menopausal women as well as in 10-20% pre-menopausal women with low estrogen levels. Vaginal atrophy is believed to be caused by low estrogen levels that result in a decrease in vaginal cell proliferation and differentiation. The decrease in cell proliferation leads to thinning of vaginal epithelium and to a lack of glycogen production by intermediate cells. Glycogen plays an important role in maintaining vaginal ecosystem by serving as food for Lactobacilli acidophilus, the normal flora in the vagina, and by serving as a substrate for acid production to maintain low vaginal pH. The thinning of vaginal epithelium and lack of glycogen in vaginal atrophy patients frequently results in vaginal dryness, discomfort and vaginitis.

While hormone replacement therapy has been used with some success to treat vaginal atrophy, new findings of the Women's Health Initiative Study indicate that such hormone replacement therapy increases the risk of heart attacks, stroke, blood clots, and breast cancer.

Therefore, a need exists for non-toxic compositions and methods to treat vaginal atrophy without the need for hormonal replacement therapies that can have negative side effects.

### SUMMARY OF THE INVENTION

The present invention is directed to compositions and methods for treating and preventing vaginal conditions characterized by low vaginal cell proliferation, low vaginal cell differentiation or low vaginal moisture vaginal atrophy. Previous work by the inventors has shown that hyaluronic acid compositions can inhibit pathogen attachment to a variety of cell types, including vaginal epithelial cells. See U.S. Ser. No. 10/401,522 and U.S. Ser. No. 10/608,848, which are incorporated herein by reference. This application illustrates that hyaluronic acid compositions can promote vaginal cell proliferation and maturation.

Therefore, in some embodiments, the invention is directed to a composition comprising an effective amount of hyaluronic acid and a pharmaceutically acceptable carrier. The compositions of the invention can promote vaginal cell growth, vaginal cell maturation and may be used to increase the thickness and restore the health of vaginal epithelium.

The invention is also directed to a method for treating or preventing a vaginal condition in a mammal. The method involves administering to a mammal an effective amount of a composition that includes a hyaluronic acid compound or a salt thereof. As illustrated herein, the hyaluronic acid compound or salt thereof can increase growth of mammalian epithelial cells and promote vaginal cell maturation. The compositions of the invention can therefore be used to treat conditions such as low vaginal cell proliferation, low vaginal cell differentiation or low vaginal moisture. In one embodiment, the condition is vaginal atrophy.

In some embodiments, the structure of the hyaluronic acid compound employed is generally that of formula I: 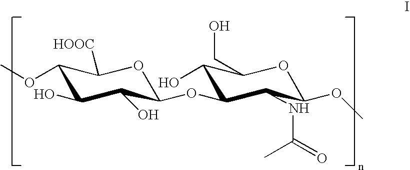
wherein n is an integer of about 1 to about 100,000. The value of n can vary.

Moreover, in many embodiments some of the individual sugar units of the hyaluronic acid can have a variety of substituents in place of the hydroxy (—OH), carboxylate (—COO⁻¹), and methylenehydroxy (—CH₂—OH) substituents that are often found in hyaluronic acid preparations. In general, the exact type of hyaluronic acid compound(s), as well as the size of the compound can vary to stimulate optimal levels of vaginal cell growth and/or maturation. Co-polymers of hyaluronic acid and other polymers are also contemplated. For example, copolymers of hyaluronic acid with other saccharide polymers, and/or non-saccharide polymers such as poly(ethylene glycol) can be employed in the compositions and methods of the invention.

The compositions of the invention are generally administered intravaginally. However, other routes are also contemplated. For example, the compositions can also be administered topically. The compositions can be incorporated into feminine products such as douches, tampons, foams, creams, sustained release implants and the like for easy use and administration. The invention further provides syringe-like applicators for administration of the compositions of the invention. Such applicators can contain a composition of the invention. Douches, tampons, foams, creams, sustained release implants and applicators can be prepared in a sterile manner to optimize the shelf life of the composition and to permit the composition to be dispensed in a sterile manner.

An effective amount of the compounds of the invention can vary, but in some embodiments the effective amount can range from about 0.01 micrograms to about 500 milligrams.

### BRIEF DESCRIPTION OF THE DRAWINGS

FIG. 1 illustrates the effect of hyaluronic acid (HLA) on cell proliferation in normal human vaginal epithelial cells in complete culture medium. Cells were treated with HLA1 (Sigma #H1751), HLA2 (Carbomer #8-01250) and HLA3 (the disaccharide, Sigma #H9649) at concentration of 100, 10 and 1 μg/ml or culture medium only (control). The symbol * represents a significant increase compared to the control group (p<0.05). Six samples were tested in each group.

FIG. 2 illustrates the effect of hyaluronic acid (HLA) on cell proliferation in normal human vaginal epithelial cells in basal culture medium without growth factors. Cells were treated with HLA1 (Sigma #H1751), HLA2 (Carbomer #8-01250) and HLA3 (a disaccharide, Sigma #H9649) at concentrations of 100, 10 and 1 μg/ml or culture medium only (control). The symbol * represents significant increase compared to the control group (p<0.05). Six samples were tested in each group.

FIG. 3 illustrates that hyaluronic acid treatment increases the observed numbers of mature vaginal epithelial cells as measured by glycogen production. Glycogen production in vaginal epithelial cell cytoplasm is a differentiation/maturation marker for vaginal epithelial cells. Each group had 5 samples and the error bar represents the standard deviation.

FIG. 4 is a bar graph illustrating that hyaluronic acid (HLA) prevents adherence of E. coli to A431 cells. The optical density at 540 nm of a 2 hr. culture of bacteria bound to A431 cells that were treated with control (PBS), 1 mg/ml HLA or 5 mg/ml HLA was used as a measure of the number of bound bacteria. The data represent mean ± SEM. N=5 in each group. The symbol * represents significant difference compared to a control group (p<0.05).

FIG. 5 illustrates that hyaluronic acid (HLA) at dosages of either 1 mg/ml or 5 mg/ml does not inhibit growth of E. coli. The graph plots optical density at 540 nm as a function of time for E. coli cultured in the presence of PBS (control), 1 mg/ml HLA or 5 mg/ml HLA.

FIG. 6 is a bar graph illustrating that hyaluronic acid (HLA) prevents adherence of Candida albicans to A431 cells. A431 cells were treated with control (PBS), 1 mg/ml HLA or 5 mg/ml HLA prior to exposure to Candida albicans. The data represent the number (mean±SEM) of yeast attached to A431 cells in each well. * represents significant decrease compared to control group (p<0.05).

FIG. 7 is a schematic diagram of one type of syringe-like applicator that can be used to deliver a hyaluronic acid composition to a vagina of a mammal. The syringe-like applicator consists of a barrel 20 and a plunger 30 with a plunger head 40. The syringe-like applicator can also have a barrier seal 60 distal to the plunger head 40 and a chamber 50 within the barrel that lies between the plunger 30 and the barrier seal 60. The chamber 50 comprises an effective amount of a hyaluronic acid compound or a salt thereof. The presence of the barrier seal 60 seals the applicator and keeps the composition contained with the applicator during shipping and handling. The barrier seal 60 can be removed by the user, or it can rupture when the user depresses the plunger. At the time of use, the applicator is inserted into the vagina and the plunger 30 is depressed. This force will push the composition out of the applicator and into the vagina.

FIG. 8 is a schematic diagram of a vaginal insert that can be used to deliver a hyaluronic acid composition to the vagina of a mammal. The vaginal consists of a tubular shaped material 110 comprising a hyaluronic acid compound and a string 120 or other attachment for retrieval or positioning of the vaginal insert within the vagina. In some embodiments, the string 120 is optional, for example, because the vaginal insert slowly dissolves or erodes to provide sustained release of hyaluronic acid. Hence, retrieval may not be needed. The syringe-like applicator illustrated in FIG. 7 mat be used for delivery of the vaginal insert.

### DETAILED DESCRIPTION OF THE INVENTION

The invention provides compositions and methods to increase vaginal cell proliferation and vaginal cell differentiation or maturation. Such compositions can promote vaginal cell growth, the development of differentiated vaginal cells and may be used to increase the thickness and restore the health of vaginal epithelium. The compositions employed by the invention are inexpensive, non-toxic and readily available. The efficacy of these compositions and methods does not rely upon hormonal replacement or substances that are not naturally found in the body. Hence, the compositions avoid the negative side effects associated with commonly used hormone replacement therapies.

Hyaluronic Acid Compounds

Compositions employed by the invention for increasing vaginal cell proliferation and maturation contain hyaluronic acid (HLA). Hyaluronic acid, also known as hyaluronan, is a natural polysaccharide found abundantly in synovial fluid and the extracellular matrix (Luo et al., in Atala & Lanza eds., Methods in Tissue Engineering, pp. 539-553, Academic Press, 2002). Hyaluronic acid is needed for the structure and organization of extracellular matrix. Because of its excellent biocompatibility and other properties, hyaluronic acid has been used in various medical applications such as in tissue engineering, drug delivery, surgery, and cosmetics. For example, hyaluronic acid compositions have been used as lubricants of prosthetic devices, as replacement fluids during eye surgery, as anti-inflammatory agents in arthritic joints, as anti-cancer agent, as phagocytic activity enhancers, as healing and angiogenesis stimulators, as scavengers of oxygen-derived toxic free radicals and as vaccine adjuvants. Hyaluronic acid therefore has a long history of use in a variety of in vivo situations.

Natural hyaluronic acid usually consists of 200-10,000 repeating disaccharide units of (β-1,4-)-linked D-glucuronic acid and (β-1,3-)-N-acetyl-D-glucosamine. In some embodiments, the structure of the hyaluronic acid compound is generally that of formula I: 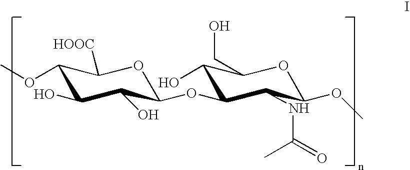
or a salt thereof, wherein n is an integer of about 1 to about 100,000. Salts of such hyaluronic acid compounds include any physiologically acceptable salt available to one of skill in art. Examples include sodium, calcium or potassium salts of hyaluronic acid.

While the molecular weight of hyaluronic acid isolated from natural sources typically ranges from about 1×10⁵ to 5×10⁶ daltons, the hyaluronic acid employed in the compositions of the invention can have a wide variety of molecular weights. For example, the molecular weight can vary from about 300 daltons to about 1×10⁸ daltons. Hence, the value of n can vary. For example, hyaluronic acid compounds can be utilized that have as few as about two to about six units (n=about 1 to about 6), as few as about two to about ten units (n=about 2 to about 10), as few as about three to about twenty units (n=about 3 to about 20), as few as about three to about thirty units (n=about 3 to about 30), or as few as about four to about one hundred units (n=about 4 to about 100). The upper limit on the number of disaccharide units (n) for the hyaluronic acid compounds used in the invention can also vary. For example, hyaluronic acid compounds can be utilized that have as many as about one hundred to about two hundred units (n=about 100 to about 200), as many as about one hundred to about five hundred units (n=about 100 to about 500), as many as about three hundred to about one thousand units (n=about 300 to about 1000), as many as about three hundred about ten thousand units (n=about 300 to about 10,000), or as many as about four hundred to about one hundred thousand units (n=about 400 to about 100,000). Mixtures of hyaluronic acid compounds with different lengths can also be used in the compositions and methods of the invention. Therefore, the length of the hyaluronic acid compounds employed in the invention can be adjusted to obtain optimal coverage of the urogenital tract or to facilitate preparation of a composition that can readily be administered to a mammal.

Moreover, in many embodiments some of the individual sugar units of the hyaluronic acid can have a variety of substituents in place of the hydroxy (—OH), carboxylate (—COO⁻), methylenehydroxy (—CH₂—OH) and N-acetyl (—NH—CO—CH₃) substituents that are often found in hyaluronic acid preparations. For example, lower alkyl moieties can replace any of the hydrogen atoms from the hydroxy (—OH), carboxylic acid (—COOH) and methylenehydroxy (—CH₂—OH) substituents of the hyaluronic acid compounds employed in the invention. Amino or lower alkyl amino groups can replace any of the OH groups on the hydroxy (—OH), carboxylic acid (—COOH) and methylenehydroxy (—CH₂—OH) substituents of the hyaluronic acid compounds employed in the invention. Sulfate (—SO₄⁻) can replace any of the OH groups on the hydroxy (—OH), carboxylic acid (—COOH) and methylenehydroxy (—CH₂—OH) substituents of the hyaluronic acid compounds employed in the invention. Hence, substituents that can be present instead of, or in addition to, the substituents shown in formula I include sulfate (—SO₄⁻), lower alkoxy, lower alkanoyloxy, and/or lower alkanoylaminoalkyl. Other examples of modified saccharide units and methods for generating such modified saccharide units are provided in Luo et al., Modification of Natural Polymers: Hyaluronic Acid, in Atala and Lanza, eds., Methods in Tissue Engineering, 539-53, Academic Press, San Diego (2002), which is incorporated herein by reference.

As used herein, lower alkyl means (C₁-C₆) alkyl. Such (C₁-C₆) alkyl can be methyl, ethyl, propyl, isopropyl, butyl, iso-butyl, sec-butyl, pentyl, 3-pentyl, or hexyl. Preferred lower alkyl groups are (C₁-C₃) alkyl including methyl ethyl, propyl, isopropyl and the like. Lower alkoxy generally means (C₁-C₆) alkoxy; such (C₁-C₆) alkoxy can, for example, be methoxy, ethoxy, propoxy, isopropoxy, butoxy, iso-butoxy, sec-butoxy, pentoxy, 3-pentoxy, or hexyloxy. Lower hydroxy alkyl refers to a hydroxy group attached to a lower alkyl or lower alkylene group (e.g. —CH₂—CH₂—OH). Lower alkanoyloxy refers to (C₂-C₆)alkanoyloxy, for example, acetoxy, propanoyloxy, butanoyloxy, isobutanoyloxy, pentanoyloxy, or hexanoyloxy. Lower (C₁-C₆) alkanoylamino can, for example, be acetamino, propanoylamino, butanoylamino, isobutanoylamino, pentanoylamino, or hexanoylamino.

In some embodiments, one or more of the sugar units of the hyaluronic acid compounds can be replaced with a different type of saccharide unit. For example, the hyaluronic acid compounds utilized in the compositions or methods of the invention can have one or more glucose, glucuronic acid, mannose, mannuronic acid, galactose, galacturonic acid, gulose, guluronic acid, fucose, xylose, N-acetylneuraminic acid, N-acetyl glucosamine or other sugar units. The number of alternate saccharide units can vary in the variant hyaluronic acid compounds used in the invention. For example, the variant hyaluronic acid compounds can have about 0% to about 50% alternate saccharide units. In other embodiments, the variant hyaluronic acid compounds can have about 0% to about 40%, or about 30%, or about 20% or about 10% alternate saccharide units. Mixtures of hyaluronic acid compounds with different substituents and sugar units can also be used in the compositions and methods of the invention. Therefore, while the hyaluronic acid compounds employed in the invention can have disaccharide units like those depicted in formula I, some variability in the types of substituents and sugar units present in the hyaluronic acid preparation employed is acceptable so long as the preparation can increase vaginal cell proliferation or maturation. Hence, the hyaluronic acid compounds of the invention can have a variety of substituents and sugar units as well as having a variety of lengths.

In other embodiments, co-polymers and mixtures of hyaluronic acid with other polymers can be employed in the compositions and methods of the invention. In some embodiments, the polymers are non-saccharide polymers. In other embodiments, the polymers can have some saccharide units or some polysaccharide component. Examples of polymers that can be used with hyaluronic acid include proteoglycans, poly(ethylene glycol)/ poly(ethylene oxide), poly(vinyl alcohol), poly(vinylpyrrolidone), and poly(2-hydroxylethyl methacrylate). Such polymers can be covalently bonded to hyaluronic acid or simply combined with hyaluronic acid to form a mixed composition.

Proteoglycans that can be employed include aggrecans. See Alberts et al, Molecular Biology of the Cell, 1090-1112 (4^{th} ed. Garland Science, New York, 2002). Aggrecan is the major proteoglycan of cartilage and provides this tissue with its mechanical properties of compressibility and elasticity. The aggrecan molecule is composed of two N-terminal globular domains, G1 and G2, which are separated by an approximately 150 residue interglobular domain (IGD), followed by a long central glycosaminoglycan (GAG) attachment region and a C-terminal globular domain, G3. Hardingham et al. (1992) in Articular Cartilage and Osteoarthritis: Aggrecan, The Chondroitin Sulfate/Keratin Sulfate Proteoglycan from Cartilage (Kuettner et al.) pp.5-20, Raven Press, New York; Paulson et al. (1987) Biochem. J. 245, 763-772. These aggrecan molecules interact through the G1 domain with hyaluronic acid and a link protein to form large molecular weight aggregates which are trapped within the cartilage matrix. Hardingham et al. (1972) Biochim. Biophys. Acta 279, 401-405; Heinegard et al. (1974) J. Biol. Chem. 249, 4250-4256; and Hardingham, T. E. (1979) Biochem. J. 177, 237-247]. Aggrecan and other proteoglycan polymers can therefore bind to cell-surface-bound hyaluronic acids, increase cell surface coverage and/or facilitate the cellular growth or maturation of vaginal cells.

One example of an amino acid sequence for a human aggrecan is available in the NCBI database at accession number NP 037359 (gi: 6995994); a nucleotide sequence for this aggrecan is available at accession number NM 013227 (gi: 6995993). See website at ncbi.nlm.nih.gov. Another example of an amino acid sequence for a human aggrecan is available in the NCBI database at accession number NP 001126 (gi: 4501991); the nucleotide sequence for this aggrecan can be found at accession number NM 001135 (gi: 4501990). See website at ncbi.nlm.nih.gov. Other aggrecan sequences can be found in the NCBI database. See website at ncbi.nlm.nih.gov.

According to the invention, hyaluronic acid compounds, co-polymers, polymer mixtures and their salts, associate with cells, tissues and/or extracellular materials and promote vaginal cell proliferation and/or vaginal cell maturation in animals. The term “animal,” as used herein, refers generally to a warm-blooded animal. Mammals include cattle, buffalo, sheep, goats, pigs, horses, dogs, cats, rats, mice, rabbits, chickens, turkeys, and humans. Also included are other livestock, domesticated animals and captive animals.

An effective amount of a hyaluronic acid compound, co-polymer, or salt thereof, for promoting vaginal cell growth and/or maturation is an amount that increases the growth of a population of vaginal cells, for example, epithelial cells, relative to a population of the same type of vaginal cells that received no hyaluronic acid. To achieve the desired inhibition, the composition may be administered as single or divided dosages, for example, of at least about 0.001 μg to about 100 to 200 mg, of about 0.01 μg to about 75 to 100 mg, of about 0.1 μg to about 50 to 75 mg or about 1.0 μg to about 30 to about 50 mg of one or more hyaluronic acid compound, although other dosages may provide beneficial results. In some embodiments, the dosage can vary from about 1 mg to about 50 mg.

Daily doses of the compositions of the invention can vary as well. Such daily doses can range, for example, from about 0.001 mg/day to about 500 mg/day, from about 0.01 mg/day to about 250 mg/day, from about 0.1 mg/day to about 120 mg/day, from about 0.1 mg/day to about 100 mg/day, from about 0.1 mg/day to about 75 mg/day, and from about 0.1 mg/day to about 50 mg/day of one or more of the hyaluronic acid compounds.

The amount administered will vary depending on various factors including, but not limited to, the disease, the weight, the physical condition, the health, the age of the animal, and whether prevention or treatment of vaginal atrophy is to be achieved. Such factors can be readily determined by the clinician employing animal models or other test systems that are available in the art.

Several receptors exist on human cell surfaces for hyaluronic acids, such as the CD44 and RHAMM receptors. See PCT Application WO 02/24223; Goodison et al., J. Clin. Pathol: Mol. Pathol., 1999, 52, 189-196; Lesley et al., Journal of Biological Chemistry, 2000, 275, 26967-26975; Lee & Spicer, Current Opinion in Cell Biology, 2000, 12, 581-586; Sackman & Bruisma, Chem. Phys. Chem., 2002, 3, 262-269. The CD44 receptor is a ubiquitously expressed family of cell surface adhesion proteins. Cells not expressing CD44 usually have other similar receptors. For example, lymphatic vessel endothelial cells have LYVE proteins that may bind hyaluronic acids. While the hyaluronic acid compounds of the invention may bind to such receptors, the efficacy of the compositions and methods of the invention does not necessarily depend upon such binding.

Methods of Use

The present invention is directed to methods of treating or preventing or otherwise ameliorating vaginal conditions characterized by poor vaginal cell growth, low vaginal moisture and poor vaginal cell differentiation. The compositions of the invention can also be used in vaginal moisturizers and in methods for moisturizing the vagina to relieve vaginal dryness and restore health to the vaginal epithelium.

In some embodiments, the compositions and methods of the invention are used in subjects with an abnormally thin vaginal lining or subjects with an abnormally thin vaginal mucosa. Symptoms resulting from the abnormally thin vaginal lining or mucosa include vaginal dryness, discomfort, itching, dyspareunia, infection, inflammation, ulcers, discharge, and bleeding. In some instances, the compositions and methods of the invention are used to treat vaginal atrophy.

Vaginal atrophy is a condition occurring in some women, typically postmenopausal women, in which there is significant thinning of the mucosa of the vagina. The thin vaginal mucosa lacks maturation, meaning that it consists of numerous parabasal cells and little or no superficial and intermediate cells, which results in decreased glycogen deposits and a higher pH. Vaginal atrophy is caused chiefly by an estrogen deficiency because the mucosa of the vagina is an estrogen sensitive tissue and a well-known target organ for estrogen. As provided herein, vaginal atrophy and the effects of low estrogen on the vagina can be reversed by treatment with the compositions and methods of the invention.

These methods include administering to the animal an effective amount, for example, a therapeutically effective amount of hyaluronic acid. According to the invention, a therapeutically effective amount of hyaluronic acid can increase vaginal epithelial cell growth, increase the percentage of differentiated or mature vaginal epithelial cells and increase vaginal moisture.

Treatment of, or treating, a vaginal condition is intended to include the alleviation of or diminishment of at least one symptom typically associated with the vaginal condition, for example, at least one symptom typically associated with vaginal atrophy. The treatment also includes alleviation or diminishment of more than one symptom. Ideally, the treatment cures, e.g., substantially eliminates symptoms associated with the vaginal condition.

Administration

The compositions of the invention can be administered for prophylactic, therapeutic, and/or hygienic use. Such administration can be topical, vaginal, rectal, transdermal, and other convenient routes. Administration can be directly to epithelial cell surfaces. For example, the compositions of the invention can be administered directly to mucosal surfaces. Mucosal surfaces include vaginal, urogenital, rectal and the like. Surfaces of the urogenital tract that can be treated with the compositions and methods of the invention include rectal, urethral, ureteral, vaginal, cervical, uterine, etc. In some embodiments, the epithelial cell surface is vaginal.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. For prevention of certain conditions or diseases (e.g. vaginal atrophy), administration of the compositions of the invention may be essentially continuous over an indeterminate period of time, for example, at regular intervals for life. Alternatively, the compositions of the invention can be administered continuously for a pre-selected period of time or in a series of spaced doses. Local administration is generally contemplated.

The compositions are prepared by combining the active ingredients in the appropriate concentrations. Other active or inactive agents selected by one of skill in the art can optionally be added. The absolute weight of a given active agent included in a unit dose can vary widely.

The compositions of the invention can be administered in the form of an article or carrier such as a bandage, insert, syringe-like applicator, tablet, suppository, pessary, powder/talc or other solid, solution, liquid, spray, aerosol, douche, ointment, tampon, foam, cream, gel, paste, microcapsules, vaginal sponge, vaginal ring, controlled release formulation, sustained release formulation or bioadhesive gel (e.g., a mucoadhesive thermogelling composition (see, for example, U.S. application Ser. No. 10/135,805, filed on Apr. 30, 2002, which is incorporated herein by reference)).

For intravaginal administration, the therapeutic agents may be formulated as is known in the art for direct application to the vaginal area. Forms chiefly conditioned for vaginal application take the form, for example, of creams, milks, gels, dispersions, micro-emulsions, lotions thickened to a greater or lesser extent, impregnated pads, ointments, aerosol formulations (e.g., sprays or foams), creams, lotions, pastes, jellies, sprays, and aerosols. Alternatively, the composition can be formulated to be part of an adhesive polymer, such as polyacrylate or acrylate/vinyl acetate copolymer.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays are conveniently delivered from pressurized packs, for example, via a specially shaped closure. The active compositions can also be delivered via iontophoresis, e.g., as disclosed in U.S. Pat. Nos. 4,140,122; 4,383,529; or 4,051,842. The percent by weight of a prophylactic agent of the invention present in a formulation will depend on various factors, but generally will be from about 0.01% to about 98% of the total weight of the formulation, and typically about 0.1 to about 90% by weight.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions with a pH of about 4.5 to about 5.5.

Furthermore, the active ingredients may also be used in combination with other therapeutic agents, for example, anti-microbial agents, pain relievers, anti-inflammatory agents, vitamins (e.g., vitamin B, C or E), aloe vera and the like, whether for conditions described herein or some other condition.

The present invention further pertains to a packaged pharmaceutical composition for controlling or preventing vaginal atrophy such as a kit or other container. The kit or container holds a therapeutically effective amount of a pharmaceutical composition for preventing, controlling or inhibiting vaginal atrophy and instructions for using the pharmaceutical composition for prevention, control or inhibition of vaginal atrophy. The pharmaceutical composition includes a composition of the present invention, in a therapeutically effective amount such that vaginal atrophy is prevented, controlled or inhibited.

In addition, the invention provides a vaginal insert that can release the hyaluronic acid compounds in a controlled fashion. Such a vaginal insert can be biodegradable or non-biodegradable. The vaginal insert provides sustained release of the active ingredients at an appropriate rate for achieving the desired degree of vaginal cell growth or of the desired degree of vaginal atrophy prevention or treatment.

In some embodiments, the active ingredients can be formulated with oleaginous bases or ointments to form a semisolid composition with a desired shape. For example, the composition can be shaped for easy insertion into an orifice such as the vagina. This class of formulations comprises the active ingredients and hydrocarbon-based semisolids containing dissolved and/or suspended bacteriostats/preservatives and a buffer system. The petrolatum component in these bases can be any paraffin ranging in viscosity from mineral oil employing incorporated isobutylene, colloidal silica, or stearate salts to paraffin waxes. White and yellow petrolatums are examples of such systems. Bases of this class can be made by incorporating high-melting waxes into a fluid mineral oil via fusion or by incorporation of polyethylene into mineral oil at elevated temperature. Polysiloxanes (also known as silicones) are suitable for use in these bases and typically have a viscosity in the range of about 0.5 to 10⁶ centistokes. The organic entities attached to the polysiloxane are preferably lower molecular weight hydrocarbon moieties having from 1 to 8 carbons each, such as lower alkyl, lower alkenyl, phenyl and alkyl substituted phenyl, and phenyl(lower)alkyl, such as benzyl. In such a moiety, each lower alkyl or alkenyl group preferably has 1 to 3 carbons inclusive, such as in a dimethylsiloxane polymer.

Absorption bases can be used with such an oleaginous system. In addition to the active ingredients, additional ingredients with the capacity to emulsify a significant quantity of water are employed. Water-in-oil (w/o) emulsions can be formed wherein the external phase is oleaginous in character.

Preservatives/bacteriostats, such as the parabens, buffer systems, etc. can be incorporated into these bases as emulsified aqueous solutions together with the active ingredient. Diverse additives are conveniently used as the emulsifier, and these include, but are not limited to, cholesterol, lanolin (which contains cholesterol and cholesterol esters and other emulsifiers), lanolin derivatives, beeswax, fatty alcohols, wool wax alcohols, low HLB (hydrophobe/lipophobe balance) emulsifiers, and assorted ionic and nonionic surfactants, singularly or in combination.

Water-In-Oil emulsion bases can be employed in the compositions, inserts and articles of the invention. These formulations can be an expansion of the general class of absorption bases that includes liquids or creams. They can be prepared by taking a mixture of the active ingredients with oil phase ingredients, bacteriostats/preservatives and buffer salts that are dissolved or suspended therein and to which water has been added to form a water-in-oil emulsion.

Oil-In-Water emulsion bases can also be utilized in the compositions, inserts and articles of the invention. These systems are semisolid emulsions, micro-emulsions, or foam emulsion systems. Usually such a system has a “creamy white” appearance. Typically, the internal oil phase is in the range in percentage composition of about 10% to about 40% oil by weight and the external phase may contain 80% or more water. The oleaginous phase may contain, but is not limited to, long-chain alcohols (cetyl, stearyl), long-chain esters (myristates, palmitates, stearates), long-chain acids (palmitic, stearic), vegetable and animal oils and assorted waxes. These can be made with anionic, cationic, nonionic or amphoteric surfactants, or with combinations especially of the nonionic surfactants. The examples below are exemplary of these systems, but those skilled in the art will appreciate that substitutions and additions or omissions of the specified components could be made by one who is skilled in the art.

Inserts and suppositories containing the active ingredients can be, for example, oleaginous in nature that melt at body temperature, or polyethylene glycol-based compositions that dissolve in mucosal (e.g. vaginal) fluids. Additional bases for suppositories are glycerin and glycerinated gelatin.

The active ingredients can also be formulated into articles, transdermal patches, bandages, wound dressings and inserts using buffered gels made with gelling agents. Some examples of these gelling agents are: cellulosics, cationic polymers, polyoxyalkylenes, and carboxyvinyl polymers. Cellulosics useful in the formulations of the invention include, for example, methyl cellulose, carboxymethyl cellulose, hydroxyethyl cellulose, and hydroxypropyl cellulose. Cationic Polymers useful in the formulations of the invention include “Polyquaternium-10”, a polymeric quaternary ammonium salt of hydroxyethyl cellulose reacted with a trimethyl ammonium-substituted epoxide, and the like. Polyoxyalkylenes useful in the invention include polyoxyethylene-polyoxypropylene esters of lanolin and derivatives thereof. Carboxyvinyl polymers useful for the formulations of the invention include cross-linked acrylic acid polymers, e.g., those commercially available from B. F. Goodrich Co., Akron, Ohio, under the designation CARBOPOL™.

Controlled or sustained release can be achieved by the addition of time-release additives, such as polymeric structures, matrices, that are available in the art. For example, the compositions of the invention may also be administered through the use of hot-melt extrusion articles, such as bioadhesive hot-melt extruded film (see, for example, U.S. Pat. No. 6,375,963, which is incorporated herein by reference). The formulation can comprise a cross-linked polycarboxylic acid polymer formulation, generally described in U.S. Pat. No. 4,615,697 to Robinson (hereinafter “the '697 patent”), which is incorporated herein by reference. In general, about eighty percent of the monomers of the polymer in such a formulation contain at least one carboxyl functionality. The cross-linking agent should be present at such an amount as to provide enough adhesion to allow the system to remain attached to the target epithelial or endothelial cell surfaces for a sufficient time to allow the desired release of hyaluronic acid to take place.

An insert or article can comprise a mixture or coating of polymers that provide release of the active agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients. Such a water-soluble pore-forming agent can be polyethylene glycol, polypropylene glycol, a mixture or polymer of sugars (lactose, sucrose, dextrose, etc.), salts, poloxamers, hydroxypropylcellulose, polyvinyl alcohol and other water-soluble food grade and other excipients.

When PEG is used as a pore forming agent, the molecular weight of PEG is in the range from about 200 to about 20,000, alternatively, from about 400 to about 8,000. For example, PEG having a molecular weight of about 540 to about 8,000 is used. In another embodiment, the PEG has a molecular weight of about or above 1,000 to about 8,000. The molecular weight of PEG used for the coating with the formulation of the invention will depend on the ability of PEG to form a coating film that is non-sticky, having enough strength and creating adequate pore size for controlling the release of active ingredients over the desired time period both in vitro and in vivo.

The pore-forming agent is used in the formulation of the invention in the amount effective to regulate the release of a hyaluronic acid compound at a desired rate. Preferably, the effective amount of the pore-forming agent provides long term delivery of the active agent thus increasing the useful life of a sustained-release insert, article or implant. The effective amount of the pore forming agent will depend on the desired rate and duration of the release and the ability to form a continuous microporous film during the coating process. To enable release duration over longer periods of time PEG with higher molecular weights is used. For example, PEG 8000 can provide release over a period of time that is longer than 100 days, when used in a concentration from 10 to 50%, preferably from 20 to 45% and most preferably from 30 to 45%. The concentration of PEG is expressed herein in % weight per dry basis and represents the concentration of PEG in the coating film after drying. Similarly, the thickness of the coating film is from 5 to 50 μm, preferably 30 from 10 to 30 μm and most preferably from 15 to 25 μm.

A good correlation exists between the dissolution rate of active agents and the amount of pore forming agent incorporated in the coating film based on in vitro and in vivo studies. Depending on the desired length of release, the PEG concentration ranges can be adjusted as needed. For example, in vivo duration of a coated insert may be predicted simply from the in vitro dissolution rate of the active agent at the 120-hour time point.

The inserts and articles of the invention may also comprise a water insoluble polymer. Examples of such polymers are ethylcellulose, acrylic resins, co-polymer of methacrylic acid and acrylic acid ethyl ester, polylactic acid, PLGA, polyurethane, polyethylene vinyl acetate copolymer, polystyrene-butadiene copolymer and silicone rubber, or mixtures thereof. For example, polymers sold under trade names Aquacoat ECD 30 and Eudragit RS 30 and NE 30D (registered trademarks of Rhom Tech, Inc.) can be used.

A polymer suitable for use in this invention is a polymer that is capable of forming a continuous coating film during the process of spraying and drying with active agents of the invention. The rate controlling film prepared with such a polymer is stable during implantation. The film should have enough strength to withstand tear and inner osmotic pressure, and have the stability not to swell or hydrate during the implantation life.

In one embodiment, the coating formulation of the invention is used to coat pellets comprising the active ingredients that are compressed to form a solid, biodegradable insert and then administered for promoting vaginal cell proliferation or maturation.

A polymer formulation can also be utilized to provide controlled or sustained release. Such a polymer formulation can be adjusted to control the release rate of the hyaluronic acid by varying the amount of cross-linking agent in the polymer. Suitable cross-linking agents include divinyl glycol, divinylbenzene, N,N-diallylacrylamide, 3,4-dihydroxy-1,5-hexadiene, 2,5-dimethyl-1,5-hexadiene and similar agents.

One example of a polymer for use in such a formulation is Polycarbophil, U.S.P., which is commercially available from B. F. Goodrich Specialty Polymers of Cleveland, Ohio under the trade name NOVEON™-AA1. The United States Pharmacopeia, 1995 edition, United States Pharmacopeial Convention, Inc., Rockville, Md., at pages 1240-41, indicates that polycarbophil is a polyacrylic acid, cross-linked with divinyl glycol.

Other useful bioadhesive polymers that may be used in such a drug delivery system formulation are mentioned in U.S. Pat. No. 4,615,697. For example, these include polyacrylic acid polymers cross-linked with, for example, 3,4-dihydroxy-1,5-hexadiene, and polymethacrylic acid polymers cross-linked with, for example, divinyl benzene. Typically, these polymers would not be used in their salt form, because this would decrease their bioadhesive capability. Such bioadhesive polymers may be prepared by conventional free radical polymerization techniques utilizing initiators such as benzoyl peroxide, azobisisobutyronitrile, and the like. Exemplary preparations of useful bioadhesives are provided in the '697 patent.

For vaginal administration, the formulation preferably remains attached to the epithelial or endothelial cell surfaces for a period of at least about eight to about forty-eight hours. Such results may be measured clinically over various periods of time, by testing samples from the vagina for hyaluronic acid. Such bioadhesion can be attained with bioadhesive polymers using a cross-linking agent that is present at about 0.1 to 6.0 weight percent of the polymer, with about 1.0 to 2.0 weight percent in some embodiments, to achieve an appropriate level of bioadhesion. Bioadhesion can also be measured by commercially available surface tensiometers utilized to measure adhesive strength.

The formulation may be in the form of a gel, cream, tablet, capsule, suppository, film, or any other pharmaceutically acceptable form that is tolerated by epithelial cells (e.g. the mucosa) and does not wash away easily. Different formulations are further described in U.S. Pat. No. 4,615,697, which is incorporated herein by reference.

As will be apparent to those skilled in the art, the composition of the formulation can be varied to affect certain properties of the formulation. For example, the viscosity can be varied by varying the concentration of hyaluronic acid or by adding a polymer or gel former. In some embodiments, a bioadhesive polymer can be included at various concentrations to provide greater or lesser bioadhesion. A pH sensitive bioadhesive can be utilized to effect greater release at certain pH values. The particular bioadhesive qualities should prevent the composition from being diluted or washed away, thereby increasing the utility of the present formulation.

Liquid compositions of the invention can be administered from absorbent materials, such as a bandage, tampon or sponge, or as a spray/aerosol (applied to the affected area using a pump-type or aerosol sprayer). The use of a tampon, in which the composition of the invention has been incorporated, is advantageous in that it the composition will be slowly and continuously released even though it may be continuously carried away by menstrual blood or other vaginal discharge. Providing the composition in the form of a solution, which may initially be provided in a concentrated liquid form, or as a dissolvable powder, tablet or the like requiring the addition of water, saline or other suitable diluents prior to use, enables the composition to be administered as a vaginal douche.

Solid compositions of the invention can be applied by any number of means, including the use of applicators or by patient self-insertion. For example, creams, lotions, suppositories, foams, pastes, ointments, gels, tablets, or tampons may be administered using an applicator, such as a squeeze-type or plunger-type applicator. Administering the composition as a suppository is advantageous as it provides convenience, ease of application, increased safety and/or neatness. Administering the composition as a cream having low surface tension is advantageous as it provides a uniform wetting action that assists in composition penetration into crypts and crevices of the wound or the orifice. Such a creamy composition can also act as a moisturizer.

Additionally, additives may be mixed in with the formulation for maximum or desired efficacy of the delivery system or for the comfort of the patient. Such additives include, for example, lubricants, plasticizing agents, preservatives, gel formers, tablet formers, pill formers, suppository formers, film formers, cream formers, disintegrating agents, coatings, binders, vehicles, coloring agents, taste and/or odor controlling agents, humectants, viscosity controlling agents, pH-adjusting agents, and similar agents.

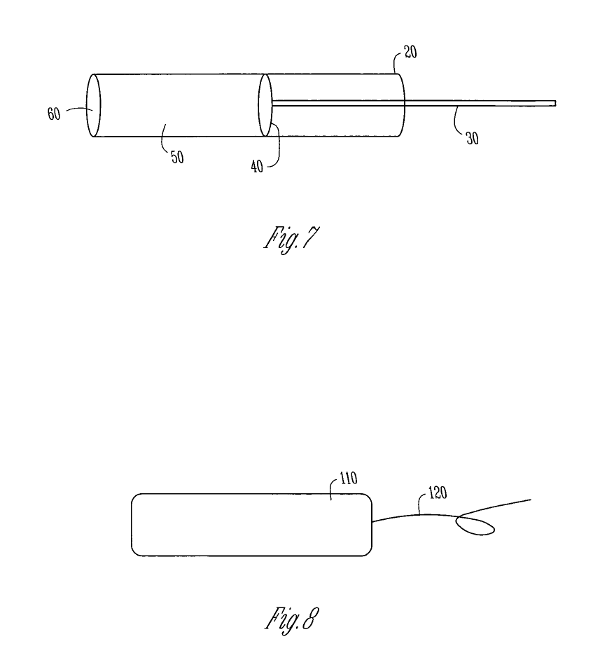

One desirable embodiment provides for compositions of the invention in a syringe-like applicator (also known as a plunger-type or syringe-like applicator (see FIG. 7)). For example, a composition including hyaluronic acid may be placed into a chamber 50 within the barrel 20 of a syringe-like applicator. The chamber is sealed at the distal end with a barrier seal 60 and at the proximal end by the plunger head 40. The presence of the barrier seal 60 seals the applicator and keeps the composition contained with the applicator during shipping and handling. However, the barrier seal 60 can be removed by the user or it can rupture when the user depresses the plunger 30. At the time of use, the applicator is inserted into the vagina and the plunger 30 is depressed. This force will push the composition out of the applicator and into the vagina. As an alternative, a tapered tip can be used in place of the barrier seal 60. One embodiment of the invention provides an aqueous gel containing a mucoadhesive material, such as carboxymethylcellulose (optionally mixed with a thermogelling mucoadhesive agent), to be mixed with hyaluronic acid to thereby form a composition of the invention. An additional embodiment provides for the encapsulation of hyaluronic acid in polymeric microparticles. Once in situ, the polymer dissolves and the hyaluronic acid is released. In this case, release of hyaluronic acid can be controlled by the microparticles to provide extended production of the desired product (e.g., sustained release). The delivery vehicle is not limited to use in the vagina, but could also be applied to a wide variety of biomedical applications where delivery of hyaluronic acid is desired. Appropriate modification of the delivery vehicles described herein is within the skill of those in the art.

Additionally, the composition and/or delivery materials may contain additional beneficial agents that can improve the health of the vagina. For example, polymers used as carrier or for encapsulation or for sustained release may be hydrolytically degraded into an acid or acid producing species. One such polymer is a poly (vinyl alcohol) backbone with pendant polycaprolactone chains that, upon disintegration, yields poly [vinyl (polycaprolactate)]. The polycaprolactone is hydrolytically degraded into caproic acid. This acid aids in lowering pH and controlling harmful bacterial growth, thus helping to restore balance to the epithelium. In addition, this material is melt processable and can be formed into a system for controlled delivery of the hyaluronic acid. Additionally, a peroxide of Laureth-4 (e.g., a Laureth-4 terminal peroxide) would release laureth-4 and peroxide (e.g., hydrogen peroxide). Laureth-4 decreases TSS-1 production by S. aureus and the peroxide is available to suppress undesirable anaerobes and Gardnerella vaginalis, thus reducing toxin production while reestablishing the vaginal flora.

The Examples further illustrate certain aspects of the invention and are not intended to limit the invention in any manner.

### EXAMPLE 1

### Hyaluronic Acid Promotes Vaginal Cell Proliferation

Materials and Methods

Normal human vaginal epithelial cells from Clonetics (NHVE 5164) were subcultured into 96 well plates at 37° C., 5% CO₂. Cells were cultured with basal PrEBM (Clonetics CC 3165) supplemented with cell growth medium bulletKit (Clonetics, cc-3166). Cells were treated with HLA having different molecular weights (MW), HLA1 (Sigma #H1751, intrinsic viscosity=100), HLA2 (MW, 150-500 k, Carbomer #8-01250) and HLA3 (a disaccharide, MW 405, Sigma #H9649). HLA was dissolved in either basal culture medium or basal medium supplemented with growth factors (complete medium) at concentration of 100, 10 or 1 μg/ml. Control group cells were treated with culture medium only. Each treatment group included 6 samples. The media for the different samples were changed 48 hours later and cell proliferation was examined at 72 hours by using CellTiter 96 Aqueous One Solution from Promega (#TB245). Twenty μl of the reagent was added to each well. The plates were returned to cell culture incubator for 3 hours. The absorbance in each well was measured at 490 nm with a microtiter reader.

Results

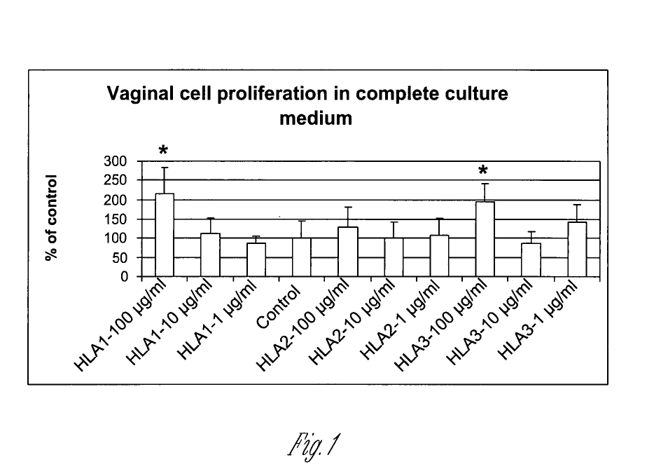

As shown in FIG. 1, when the vaginal cells were cultured in complete medium, HLA with high MW (HLA 1) and the dimmer (HLA 3) at concentration of 100 μg/ml significantly promoted vaginal cell proliferation while low concentrations, 10 and 1 100 μg/ml had no significant effect on cell proliferation compared to control group. HLA2 had no statistically significant effect on the vaginal cell proliferation at any of the three concentrations tested.

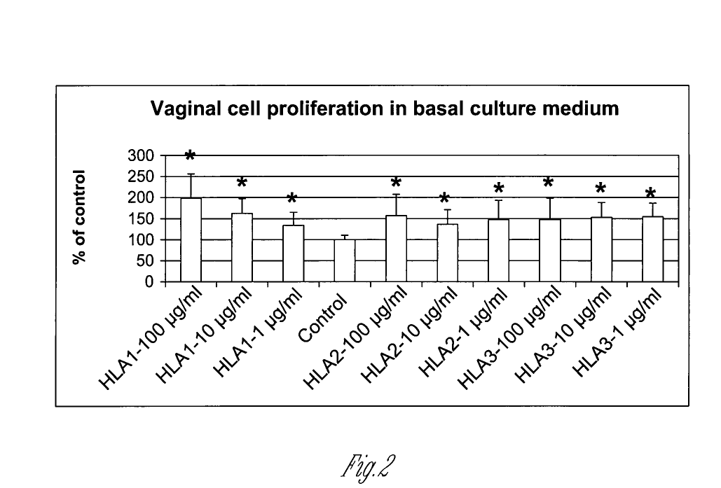

However, all of the forms of HLA exhibited stimulatory effects at all the three concentrations tested in basal culture medium containing no growth factors (FIG. 2).

These results indicate that HLA can stimulate cell proliferation. Vaginal atrophy is the thinning of the vaginal epithelium that may be caused by low estrogen levels resulting in a decrease in vaginal cell proliferation. Atrophic vaginitis, the overgrowth of pathogens due to the thinning of protective epithelium, can evolve in patients having vaginal atrophy. As shown in previous applications by the inventors, hyaluronic acid can inhibit pathogen attachment. See U.S. Ser. No. 10/401,522 and U.S. Ser. No. 10/608,848, which are incorporated herein by reference. Hence, compositions containing HLA that stimulate vaginal cell proliferation may be used to increase the thickness of vaginal epithelium, prevent pathogen attachment and restore healthy vaginal epithelium.

### EXAMPLE 2

### Hyaluronic Acid Promote Glycogen Production of Human Vaginal Cells

This Example illustrates that hyaluronic acid treatment increases glycogen production in human vaginal cells. Glycogen is produced by the vaginal epithelial cells of intermediate epidermal layers. A decrease in glycogen content is one of the most striking changes in the postmenopausal vaginal epithelium, mainly due to the fact that cells in the parabasal layers fail to further differentiate. Therefore, glycogen content in the vaginal epithelial cell cytoplasm has been used as a differentiation/maturation marker for vaginal epithelial cells.

Primary vaginal epithelial cells were cultured in 96-well plates. Five samples were tested for each group. Treatment was for 5 days with culture medium solutions (PrEBM, Clonetics CC-3165) without (the control group) and with 0.1 mg/mL hyaluronic acid (Sigma H-1751). The media were changed every other day. At the end the treatment, the media was removed, and the well was washed twice with HBSS.

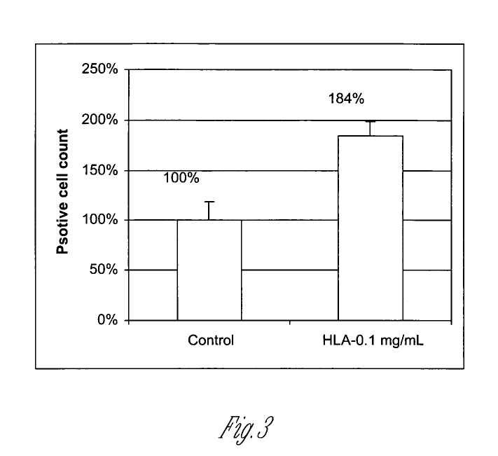

Glycogen tests were performed using a periodic acid Schiff (PAS) assay as described below. Before the test, cells were fixed with a 10 vol % Formalin/ethanol solution for 1 hour at room temperature, followed by continuous rinsing with deionized (DI) water for 1 minute. Harleco® PAS Aassay Kit (from EM Science 64945/43) was used for glycogen detection. The basic principle is that the hydroxy groups on the glycogen units were first oxidized into aldehyde groups by periodic acid, and the aldehyde groups reacted with Schiff's agent to produce a red colorant. The specific assay operations were all done at room temperature as follows:
1. Periodic acid reagent was added to the well/chamber and allowed to stand for 10 minutes).
2. Samples were continuously rinsed in water for one minute.
3. Schiff's reagent was added and allowed to stand for 15 minutes.
4. Samples were continuously rinsed in deionized water for one minute.
5. Sodium carbonate reagent (diluted 2× with deionized water) was added and allowed to stand for 5 minutes.
6. Samples were continuously rinsed in deionized water for 5 minutes.
7. Light Green SF Yellowish reagent was added and samples were allowed to stand for 30 seconds.
8. Samples were rinsed in deionized water briefly for 10 seconds.
9. Samples were dehydrated with a series of ethanol/water mixtures (75%, 90%, 95% to 100% ethanol) and xylene.
10. Samples were mounted with mounting medium.
   The numbers of glycogen-stained positive cells were manually counted under microscope. These results are shown in FIG. 3. The observed increase in glycogen production was statistically significant (p<0.05). Hence, hyaluronic acid compositions can promote maturation of vaginal epithelial cells.

### EXAMPLE 3

### Hyaluronic Acid Inhibits E. coli Attachment to Human Epithelial Cells

This Example illustrates that reduced numbers of E. coli attach to substrates containing hyaluronic acid.

Materials and Methods

Cultured A431 cells were used as a model human epithelial cell line. A431 cells were obtained from American Cell Type Culture Collection, catalog # CRL-1555. A monolayer of A431 cells was grown on a 24-well tissue culture plate until confluent. E. coli with P family pili, ATCC 53505, were cultured in trypticase soy broth (TSB) overnight. Phosphate buffered saline (PBS, control) or hyaluronic acid (Sigma, catalog # H1876) solutions at a concentration of 1 mg/ml or 5 mg/ml in PBS were added to the A431 cell layers, using a volume of 1.2 ml/well. For comparison, a control plate was set up and treated identically as the test plate, except that the wells in the control plate were given PBS without hyaluronic acid. After incubation for 30 minutes at 37° C., 0.5 ml of solution was removed from the wells and 0.5 ml of the bacteria suspension was added. The bacterial suspension employed had an optical density of 1.0 at 540 nm. After 60 minutes, the supernatant in each well was removed and the wells were rinsed thoroughly with PBS to remove all non-bound bacteria. Trypticase soy broth was then added to wells, and the system was incubated at 37° C. with shaking at 100 rpm/min for 2 hours. Bacterial concentration was measured by the detection of optical density of bacterial suspension at 540 nm.

All the data are expressed as mean ± SEM and were analyzed by one-way Analysis of Variance (ANOVA) followed by a Tukey-Kramer Multiple Comparisons Test. Differences were considered significant at the level of p<0.05.

Results

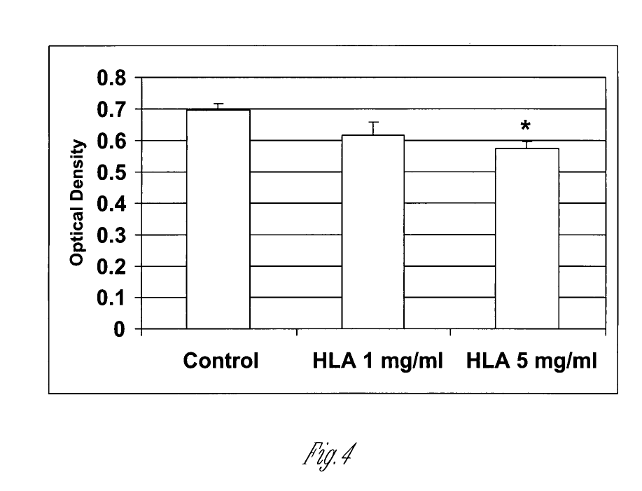

FIG. 4 graphical illustrates the results of the E. coli adhesion test. Hyaluronic acid at a concentration of 5 mg/ml significantly inhibited the number of E. coli attached to A431 cells. A concentration of 1 mg/ml hyaluronic acid had a lesser effect on E. coli adhesion. This result was repeated twice and similar effects were obtained each time.

### EXAMPLE 4

### Effect of Hyaluronic Acid on E. coli Growth

This Example illustrates that the decreased adherence of E. coli to A431 cells was not due to an inhibitory effect by hyaluronic acid on E. coli growth.

Materials and Methods

To test whether hyaluronic acid could influence cellular growth, E. coli, ATCC 53505 cells were cultured overnight in trypticase soy broth. The culture was then diluted to an optical density of 0.1 and hyaluronic acid (1 mg/ml or 5 mg/ml) in PBS was incubated with the E. coli suspension. A control comprising a suspension E. coli treated with PBS was also prepared. The test and control cells were incubated at 37° C. with shaking and the optical density of the bacterial suspensions at 540 nm was measured at 1, 2 and 3 hours after addition of the hyaluronic acid or PBS.

All the data are expressed as mean ± SEM and were analyzed by one-way Analysis of Variance (ANOVA) followed by a Tukey-Kramer Multiple Comparisons Test. Differences were considered significant at the level of p<0.05.

Results

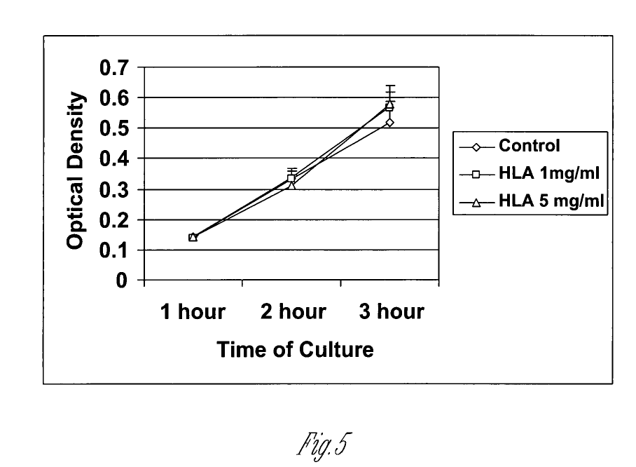

The results on the effect of hyaluronic acid on growth of E. coli are shown in FIG. 5. As illustrated in FIG. 5 neither of the hyaluronic acid solutions had any effect on bacterial growth. E. coli exposed to PBS (control) grew well under the experimental conditions. Growth of these control cells was similar to growth of cells exposed to hyaluronic acid at either 1 mg/ml or 5 mg/ml. These data indicate that the inhibition of E. coli attachment to A431 cells shown in Example 3 and FIG. 4 was not due to inhibition of bacterial cell growth.

### EXAMPLE 5

### Hyaluronic Acid Inhibits Attachment of Candida albicans to Human Epithelial Cells

This Example illustrates that hyaluronic acid inhibits attachment of Candida albicans to mammalian epithelial cells.

Materials and Methods

A culture of Candida albicans, ATCC 10231, was started in peptone/glucose culture medium and incubated at 37° C. with shaking for three days before the experiment. A monolayer of A431 cells, ATCC CRL-1555, was grown on a 24-well tissue culture plate until confluent. Hyaluronic acid at concentration of 1 mg/ml or 5 mg/ml in PBS was added to the wells (1.2 ml/well), and the plates were incubated for 30 minutes at 35° C. A control plate of confluent A431 cells was also prepared that received 1.2 ml/well PBS and no hyaluronic acid. After the 30 min incubation, 0.5 ml of solution was removed from the wells and 0.5 ml of yeast suspension (1×10⁶ cfu/ml) was added. After 2 hours of incubation, the supernatant was removed from the wells and the wells were rinsed thoroughly with PBS to remove all non-bound yeast. Then, 0.2 ml of 0.25% trypsin/EDTA was added to each well and the plates were incubated at 35° C. for 15 min. Trypsinization was stopped by adding 1 ml of 10% fetal bovine serum in culture medium. The trypsinized cell/yeast suspension was centrifuged and re-suspended in 1 ml of the medium. Serial dilutions of the Candida albicans/A431 cell suspension were plated on Sabouraud's dextrose agar plates and incubated overnight at 35° C. The number of colonies on each plate was counted and the number of yeast attached to the cells in each well was calculated.

All the data are expressed as mean ± SEM and were analyzed by one-way Analysis of Variance (ANOVA) followed by a Tukey-Kramer Multiple Comparisons Test. Differences were considered significant at the level of p<0.05.

Results

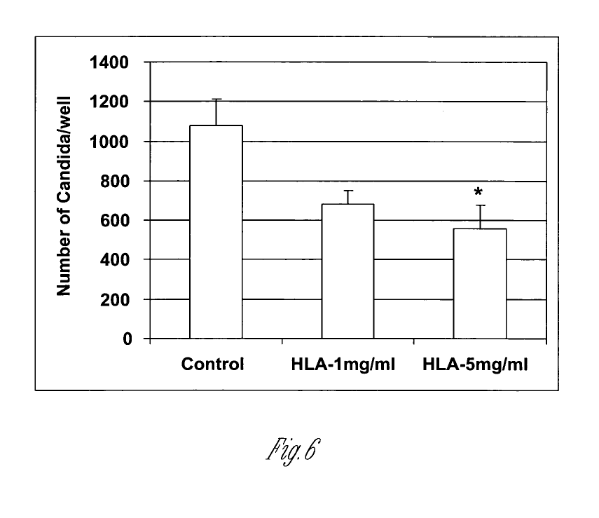

FIG. 6 summarizes the data obtained on the effects of hyaluronic acid on Candida albicans attachment to A431 cells. As shown in FIG. 6, the number of Candida albicans adhering to A431 cells was significantly inhibited by the presence of 5 mg/ml hyaluronic acid, while at a lower concentration of 1 mg/ml hyaluronic acid the effect was not so significant. This experiment was repeated and a similar result was obtained.

The results provided herein therefore demonstrate that hyaluronic acid effectively inhibits the attachment of Candida albicans and E. coli to A431 cells. This cell line has been widely used as an in vitro human epithelial cell model. The inhibitory effect of hyaluronic acid on bacterial/cell attachment was not caused by inhibition of bacterial growth.

### REFERENCES

1. P R Murray, K S Rosenthal, G S Kobayashi and M A Pfaller, Medical Microbiology (4th edition), Mosby, St. Louis, 2002.
2. A A Salyers and D D Whitt, Bacterial Pathogenesis (2nd edition), ASM Press, Washington D.C., 2002.
3. M W Hornef, M J Wick, M Shen and S Normark, Bacterial strategies for overcoming host innate and adaptive immune response, Nature Immunology, 2002, 3, 1033-1040.
4. W L Chafein, J L Lopez-Ribot, M Casanova, D Gozalbo and J P Martinez, Cell wall and secreted proteins of Candida albicans: identification, function and expression, Microbiology and Molecular Biology Reviews, 1998, 62, 130-180.
5. D Petrin, K Delgaty, R Bhatt and G Garber, Clinical and microbiological aspects of Trichomonas vaginalis, Clinical Microbiology Reviews, 1998, 11, 300-317.
6. M W Cunningham, Pathogenesis of group A Streptococcal infections, Clinical Microbiology Reviews, 2000, 13, 470-511.
7. I Muhldorfer, W Ziebuhr and J Kacker, Escherichia coli in urinary tract infections, in M Sussman edited Molecular Medical Microbiology, Chapter 81, Academic Press, San Diego, 2001.
8. S Mobastery and E F Azucena Jr., Bacterial antibiotic resistance, in Encyclopedia of Life Sciences, Nature Publishing Group, 2002.
9. C Walsh, Molecular mechanisms that confer antibacterial drug resistance, Nature, 2000, 406, 775-781.
10. M Shnayerson, The killer bug, Fortune (Industrial Edition), 2002, 146(6), 149-156.
11. S J Hultgren, C H Jones and S Normark, Bacterial adhesins and their assembly, in F C Neidhardt (editor-in-chief), Escherichia coli and Salmonella, pp. 2730-2756, ASM Press, 1996.
12. H Connell, W Agace, M Hedlund, P Klemm, M. Shembri and C Svanborg, Fimbriae-mediated adherence induces mucosal inflammation and bacterial clearance, in Khane and Ofek edited Toward Anti-adhesion Therapy for Microbial Diseases, pp. 73-80, Plenum Press, 1996.
13. N Sharon, Bacterial lectins, cell-cell recognition and infection diseases, FEBS Lett., 1987,217,145-157.
14. I Ofek and N Sharon, Adhesins as lectins: specificity and role in infection, Current Topics in Microbiology and Immunology, 1990, 151, 91-113.
15. M Mammen, S K Choi and G M Whitesides, Polyvalent interactions in biological systems: implications for design and use of multivalent ligands and inhibitors, Angew. Chem. Intl. Ed., 1998, 37, 2754-2794.
16. D Zopf and S Roth, Oligosaccharide anti-infective agents, Lancet, 1996, 347, 1017-1021.
17. A Varki, Biological roles of oligosaccharides: all of the theories are correct, Glycobiology, 1993, 3, 97-130.
18. R G Carson, A Hung, K M Schiling and S R Wu, Oral hygiene compositions containing glyceroglycolipids as antiplaques compounds, U.S. Pat. No. 5,409,902, filed on Dec. 31, 1991.
19. A Gaffar, R Gibbsons and S Tylewska, Glycoconjugate inhibition of Streptococcus pyrogenes adhesion, U.S. Pat. No. 5,401,723 filed on December 02, 1992.
20. D A Zopf, O M Simon, S Roth, E J McGuire and D H Langer, Method for inhibiting H. Pylori infection in mammalian tissue, U.S. Pat. No. 5,883,079 filed on May 12, 1998.
21. A Gaffar, R J Gibbsons and S Tylewksa, Oligosaccharide inhibition of Streptococcus pyrogens adhesion, U.S. Pat. No. 5,002,759 filed on Jul. 25, 1989.
22. P A Mardh and S Svensson, Therapeutic treatment employing oligosaccharides, U.S. Pat. No. 4,665,060 filed on Mar. 22, 1983.
23. G P Kallenius, K A Lunblad, N R Mollby, S B Svesson and J Winberg, U.S. Pat. No. 4,762,824 filed on Aug. 1, 1986.
24. Y Luo, K R Kirker and G D Prestwich, Modification of natural polymers: hyaluronic acid, in Atala and Lanza eds., Methods in Tissue Engineering, pp. 539-553, Academic Press, 2002.
25. M R Wessels and C Cywes, Prevention and treatment of streptococcal and staphylococcal infection, PCT Application WO 02/24223 filed on Sep. 21, 2001.
26. S Goodison, V Urquidi and D Tarin, CD44 cell adhesion molecules, J. Clin. Pathol: Mol. Pathol., 1999, 52, 189-196.
27. J Lesley, V C Hascall, M Tammi and R Hyman, Hyaluronan binding to cell surface CD44, Journal of Biological Chemistry, 2000, 275, 26967-26975.
28. J Y Lee and A P Spicer, Hyaluronan: a multifunctional, megaDalton, stealth molecule, Current Opinion in Cell Biology, 2000, 12, 581-586.
29. B Sackman and R F Bruisma, Cell adhesion as wetting transition?, Chem. Phys. Chem., 2002, 3, 262-269.
30. Y Cho and H Y Choi. Opportunistic fungal infection among cancer patients. A ten year autopsy study. Am. J. Clin. Pathol. 1991, 72:617-621.
31. G. Reid. Probiotics for urogenital health. Nutr Clin Care, 2002, 5(1), 3-8.
32. Y H An and R J Friedman, Concise review of mechanisms of bacterial adhesion to biomaterial surfaces, J. Biomed. Mater. Res. (Appl. Biomater.), 1998, 43, 338-348.
33. Schoor R A, Anderson B, Klumpp D J, Schaeffer. Secretory I G A differentially promotes adherence of type 1-pilliated Escherichia coli to immortalized vaginal epithelial cell lines. Urology 2001; 57:556-561.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A composition comprising an effective amount of a hyaluronic acid compound or a salt thereof, and a carrier, wherein the composition can increase growth of vaginal epithelial cells.

2. The composition of claim 1, wherein the composition can increase production of glycogen by vaginal epithelial cells.

3. The composition of claim 2, wherein the production of glycogen is a maturation marker for vaginal epithelial cells.

4. The composition of claim 1, wherein the composition is used to treat or prevent low vaginal cell proliferation, low vaginal cell differentiation or low vaginal moisture.

5. The composition of claim 1, wherein the composition is used to treat or prevent vaginal atrophy.

6. The composition of claim 1, wherein the hyaluronic acid compound or salt thereof comprises (β-1,4-)-linked D-glucuronic acid and (β-1,3-)-N-acetyl-D-glucosamine.

7. The composition of claim 1, wherein the hyaluronic acid compound or salt thereof comprises one or more glucose, glucuronic acid, mannose, mannuronic acid, galactose, galacturonic acid, gulose, guluronic acid, fucose, xylose, N-acetylneuraminic acid, or N-acetyl glucosamine saccharide units.

8. The composition of claim 1, wherein the hyaluronic acid compound or salt thereof is mixed or covalently linked to a non-polysaccharide polymer.

9. The composition of claim 8, wherein the non-polysaccharide polymer is poly(ethylene glycol), poly(vinyl alcohol), poly(vinylpyrrolidone), or poly(2-hydroxylethyl methacrylate).

10. The composition of claim 1, wherein the hyaluronic compound acid or salt thereof comprises a repeating disaccharide unit of formula I: 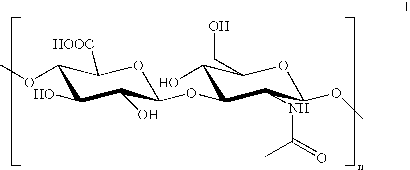
wherein n is an integer of about 1 to about 100,000.

11. The composition of claim 10, wherein a lower alkyl replaces any of the hydrogen atoms from the hydroxy (—OH), carboxylic acid (—COOH) and methylenehydroxy (—CH₂—OH) substituents within one or more of the disaccharide units of formula I.

12. The composition of claim 10, wherein an amino, a sulfate or a lower alkyl amino group replaces any of the OH groups from the hydroxy (—OH), carboxylic acid (—COOH) and methylenehydroxy (—CH₂—OH) substituents within one or more of the disaccharide units of formula I.

13. The composition of claim 1, wherein the effective amount comprises about 0.01 micrograms to about 500 milligrams of hyaluronic acid compound or salt thereof.

14. The composition of claim 1, wherein the composition further comprises an anti-bacterial, anti-fungal or anti-viral agent.

15. The composition of claim 1, wherein the composition is administered topically or intra-vaginally.

16. The composition of claim 1, wherein the composition is a lotion, cream, gel or suspension.

17. A vaginal insert for increasing vaginal cell growth in a mammal comprising an effective amount of a hyaluronic acid compound or a salt thereof, and a carrier, wherein the composition can increase growth of mammalian epithelial cells.

18. The vaginal insert of claim 17, wherein the composition can increase production of glycogen by vaginal epithelial cells.

19. The vaginal insert of claim 18, wherein the production of glycogen is a maturation marker for vaginal epithelial cells.

20. The vaginal insert of claim 17, wherein the composition is used to treat or prevent low vaginal cell proliferation, low vaginal cell differentiation or low vaginal moisture.

21. The vaginal insert of claim 17, wherein the composition is used to treat or prevent vaginal atrophy.

22. The vaginal insert of claim 17, wherein the hyaluronic acid compound or salt thereof comprises (P1-1,4-)-linked β-glucuronic acid and (β-1,3-)-N-acetyl-D-glucosamine.

23. The vaginal insert of claim 17, wherein the hyaluronic acid compound or salt thereof comprises one or more glucose, glucuronic acid, mannose, mannuronic acid, galactose, galacturonic acid, gulose, guluronic acid, fucose, xylose, N-acetylneuraminic acid, or N-acetyl glucosamine saccharide units.

24. The vaginal insert of claim 17, wherein the hyaluronic acid compound or salt thereof is mixed or covalently linked to a non-polysaccharide polymer.

25. The vaginal insert of claim 24, wherein the non-polysaccharide polymer is poly(ethylene glycol), poly(vinyl alcohol), poly(vinylpyrrolidone), or poly(2-hydroxylethyl methacrylate).

26. The vaginal insert of claim 17, wherein the hyaluronic compound acid or salt thereof comprises a repeating disaccharide unit of formula I: 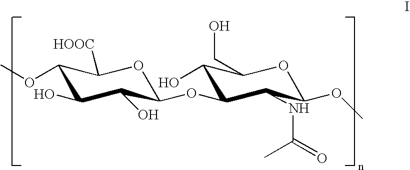
wherein n is an integer of about 1 to about 100,000.

27. The vaginal insert of claim 26, wherein a lower alkyl replaces any of the hydrogen atoms from the hydroxy (—OH), carboxylic acid (—COOH) and methylenehydroxy (—CH₂—OH) substituents, within one or more of the disaccharide units of formula I.

28. The vaginal insert of claim 26, wherein an amino, a sulfate or a lower alkyl amino group replaces any of the OH groups from the hydroxy (—OH), carboxylic acid (—COOH) and methylenehydroxy (—CH₂—OH) substituents within one or more of the disaccharide units of formula I.

29. The vaginal insert of claim 17, wherein the effective amount comprises about 0.01 micrograms to about 500 milligrams of hyaluronic acid compound or salt thereof.

30. The vaginal insert of claim 17, wherein the composition further comprises an anti-bacterial, anti-fungal or anti-viral agent.

31. A method for preventing or treating a vaginal condition in a mammal comprising administering to a mammal an effective amount of a composition comprising a hyaluronic acid compound or a salt thereof, wherein the hyaluronic acid compound or salt thereof can increase growth of mammalian epithelial cells.

32. The method of claim 31, wherein the condition comprises low vaginal cell proliferation, low vaginal cell differentiation or low vaginal moisture.

33. The method of claim 31, wherein the condition is vaginal atrophy.

34. The method of claim 31, wherein the composition can increase production of glycogen by vaginal epithelial cells.

35. The method of claim 34, wherein the production of glycogen is a maturation marker for vaginal epithelial cells.

36. The method of claim 31, wherein the composition can increase vaginal moisture.

37. The method of claim 31, wherein the hyaluronic acid compound or salt thereof comprises (β-1,4-)-linked D-glucuronic acid and (β-1,3-)-N-acetyl-D-glucosamine.

38. The method of claim 31, wherein the hyaluronic acid compound or salt thereof further comprises one or more glucose, glucuronic acid, mannose, mannuronic acid, galactose, galacturonic acid, gulose, guluronic acid, fucose, xylose, N-acetylneuraminic acid, or N-acetyl glucosamine saccharide units.

39. The method of claim 31, wherein the hyaluronic acid compound or salt thereof is mixed or covalently linked to a non-polysaccharide polymer.

40. The method of claim 39, wherein the non-polysaccharide polymer is poly(ethylene glycol), poly(vinyl alcohol), poly(vinylpyrrolidone), or poly(2-hydroxylethyl methacrylate).

41. The method of claim 31, wherein the hyaluronic compound acid or salt thereof comprises a repeating disaccharide unit of formula I: 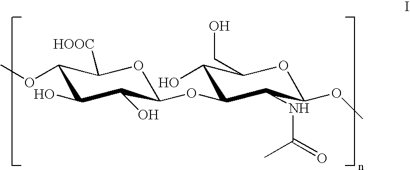
wherein n is an integer of about 1 to about 100,000.

42. The method of claim 41, wherein a lower alkyl replaces any of the hydrogen atoms from the hydroxy (—OH), carboxylic acid (—COOH) and methylenehydroxy (—CH₂—OH) substituents within one or more of the disaccharide units of formula I.

43. The method of claim 41, wherein an amino, a sulfate or a lower alkyl amino group replaces any of the OH groups from the hydroxy (—OH), carboxylic acid (—COOH) and methylenehydroxy (—CH₂—OH) substituents within one or more of the disaccharide units of formula I.

44. The method of claim 31, wherein the effective amount comprises about 0.01 micrograms to about 500 milligrams of hyaluronic acid compound or salt thereof.

45. The method of claim 31, wherein the composition further comprises an anti-bacterial, anti-fungal or anti-viral agent.

46. The method of claim 31, wherein the composition is administered topically or intra-vaginally.

47. The method of claim 31, wherein the composition is a lotion, cream, gel or suspension.
